(19) United States
(12) Patent Application Publication
(10) Pub. No.: US 2026/0327671 A1
(43) Pub. Date: Oct. 22, 2026

(54) GLUTAMIC ACID INDEPENDENT PROCESS FOR SYNTHESIS OF POLY GAMMA GLUTAMIC ACID

(71) Applicant: COUNCIL OF SCIENTIFIC AND IDUSTRIAL RESEARCH AN INDIAN REGISTERED BODY INCORPORATED UNDER THE REGN., New Delhi (IN)

(72) Inventors: Mahesh Shantappa DHARNE, Pune (IN); Pranav Girijavallabhan NAIR, Pune (IN)

(21) Appl. No.: 19/122,477

(22) PCT Filed: Oct. 19, 2023
(86) PCT No.: PCT/IN2023/050962

§ 371 Date: Apr. 18, 2025

(30) Foreign Application Priority Data
Oct. 20, 2022 (IN) 202211060179

(51) Int. Cl. C12P 21/02

(52) U.S. Cl. CPC C12P 21/02

(57) ABSTRACT

The present invention relates to a cost-effective process for the synthesis of hyper-production of poly gamma glutamic acid using L-glutamic acid independent approach. Specifically, the present invention relates to synthesis of poly gamma glutamic acid (γ-PGA) from jaggery, blackstrap molasses, and floral waste. More particularly, the present invention relates to a process for the synthesis of poly gamma glutamic acid in high yield and in the presence of Bacillus paralicheniformis NCIM 5769.

### FIELD OF THE INVENTION

The present invention relates to a process of preparing poly gamma glutamic acid. Particularly, the present invention relates to the synthesis of poly gamma glutamic acid (γ-PGA) from jaggery, blackstrap molasses, and floral waste in presence of bacterial inoculum. More particularly, the present invention relates to a process for the synthesis of poly gamma glutamic acid in higher yield in the presence of Bacillus paralicheniformis NCIM 5769 using jaggery, blackstrap molasses, and floral waste.

### BACKGROUND AND PRIOR ART OF THE INVENTION

Poly gamma glutamic acid (γ-PGA) is a naturally occurring biopolymer made from repeating units of L-glutamic acid, D-glutamic acid, or both. Owing to its biodegradable, non-toxic, and non-immunogenic properties, it has importance in the biomedical field, cosmetics, food industry, wastewater treatment, and other applications. The γ-PGA is considered a promising bio-based polymer and is already widely used in the food, medical, and wastewater industries due to its biodegradable, non-toxic, and non-immunogenic properties.

The worldwide demand for biopolymers as an attractive alternative to petro-based polymers is constantly increasing due to their phenomenal properties. The γ-PGA is one of the most expensive biopolymers with a cost of 322$ per 100 mg. However, its wider commercialization demands the need for hyper-producer strain using the low-cost substrate.

A perusal of prior literature relating to γ-PGA reveals that there are quite a few bacteria that produce poly gamma glutamic acid regardless of the addition or no addition of L-glutamic acid including B. subtilis, B. licheniformis, and B. megaterium amongst others [e.g. CN107937451A; CN104694437B; and Jing Li et al., Microb Cell Fact 21, 140 (2022)].

Even though there are potent γ-PGA producing strains available, the cost of production remains exorbitant. Thus, the only solution to this problem is to reduce the overall cost of production by using natural sources that can replace all the essential nutrients required for γ-PGA production. To achieve this, one should have a profound knowledge of various nutritional factors that affect γ-PGA production. Contrastingly, as there is enormous potential in India as well as in European countries for the valorization of surplus waste, it may be possible to direct the waste for economic and feasible production of γ-PGA.

The γ-PGA producers can be classified on the basis of the external supply of L-glutamic acid in the fermentation medium as L-glutamic acid-dependent producers and L-glutamic acid-independent producers. The L-glutamic acid-dependent producers have an advantage of high γ-PGA production compared to independent producers; however, the cost of production is relatively higher due to the external supply of L-glutamic acid. Whereas in L-glutamic acid independent producers, the γ-PGA yield is comparatively lower, and the production cost is also less. Therefore, the concept of the “Minimum nutrient with maximum yield” is the key to the successful commercialization of γ-PGA i.e., addition of the least possible nutrient in the fermentation medium to obtain maximum yield of γ-PGA.

Therefore, there is a need in the art to reduce the overall production cost of γ-PGA along with increasing the yield of highly pure poly gamma glutamic acid (γ-PGA) from inexpensive substrates.

### OBJECTIVES OF THE INVENTION

The objective of the present invention is to provide a process for the synthesis of highly pure poly gamma glutamic acid (γ-PGA) from inexpensive substrates.

Another objective of the present invention is to provide a process for the synthesis of γ-PGA from waste materials.

An important objective of the present invention is to provide a process for the synthesis of γ-PGA from jaggery, blackstrap molasses, and floral waste, which are inexpensive materials, and their use valorize surplus waste into value-added commodity polymers.

Yet another objective of the present invention is to reduce the production cost of poly gamma glutamic acid (γ-PGA).

Yet another object of the present invention is to produce poly gamma glutamic acid (γ-PGA) at maximum yield by using the glutamic acid-independent approach.

### SUMMARY OF THE INVENTION

Accordingly, to accomplish the objectives of the invention, the present invention provides a process for synthesis of poly gamma glutamic acid without using L-glutamic acid by using natural materials such as jaggery, blackstrap molasses, and floral waste.

In an aspect, the present invention relates to a process for the preparation of poly gamma glutamic acid using natural source and cost-effective sources including the step of:
a) introducing a natural source into a fermentation medium comprising a nitrogen source and other suitable components in a fermenter;
b) adding a bacterial inoculum to the above fermentation medium to obtain a fermentation medium mixture;
c) incubating fermentation medium mixture of step b) followed by centrifugation to obtain a mixture; and
d) adding ice-cold solvent to the mixture of step c) to precipitate the poly gamma glutamic acid product.

In specific aspect of the present invention, the natural source is selected from flower waste, jaggery, black strap molasses, or their combinations and the like.

In specific aspect of the present invention, the bacteria used for the synthesis of poly gamma glutamic acid is selected from Bacillus species and more particularly Bacillus species is selected from Bacillus anthracis, Natrialba aegyptiaca, Bacillus subtili, Bacillus licheniformis, Bacillus megaterium, Staphylococcus epidermidis, Bacillus siamensis, and Bacillus paralicheniformis.

In an aspect, the present invention relates to glutamic acid independent process for the synthesis of poly gamma glutamic acid from inexpensive substrate in a batch fermentation mode.

Various objects, features, aspects and advantages of the inventive subject matter will become more apparent from the following detailed description of preferred embodiments. These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

The following drawings form a part of the present specification and are included to further illustrate aspects of the present disclosure. The disclosure may be better understood by reference to the drawings in combination with the detailed description of the specific embodiments presented herein.

FIG. 1 represents the effect of concentration of the jaggery on the yield of poly glutamic acid of A1 to A5 in the process of present invention, in accordance with an embodiment of the present disclosure.

FIG. 2 represents the effect of increased concentration of jaggery on the yield of γ-PGA production of B1 to B5 in the process of present invention, in accordance with an embodiment of the present disclosure.

FIG. 3 depicts the experimental design for minimum nutrient and maximum yield concept for PGA production from jaggery, in accordance with an embodiment of the present disclosure.

FIG. 4 shows the effect of increased concentration of ammonium nitrate on the yield of γ-PGA production from jaggery of the present invention, in accordance with an embodiment of the present disclosure.

FIG. 5 depicts the experimental design for the “minimum nutrient with maximum yield” concept for PGA production from molasses, in accordance with an embodiment of the present disclosure.

FIG. 6 shows the effect of ammonium nitrate on L-glutamic acid independent production of γ-PGA from molasses of the present invention, in accordance with an embodiment of the present disclosure.

FIG. 7 represents the highly viscous fermented broth of γ-PGA produced from molasses passing the inversion test, in accordance with an embodiment of the present disclosure.

FIG. 8 shows the effect of concentration of floral waste on the yield γ-PGA production of the present invention, in accordance with an embodiment of the present disclosure.

### DETAILED DESCRIPTION OF THE INVENTION

The invention will now be described in detail in connection with certain preferred and optional embodiments, so that various aspects thereof may be more fully understood and appreciated. The present invention relates to the glutamic acid independent process for the synthesis of poly gamma glutamic acid.

For the purpose of this invention the term “glutamic acid independent process” for the synthesis of poly gamma glutamic acid should be construed as the process which is carried out without addition of external glutamic acid.

In an embodiment, the present invention relates to a process for preparation of poly gamma glutamic acid using natural sources and cost-effective sources including the steps of:
a) introducing 20-60 wt. % of natural sources into a 50-1000 mL fermentation medium comprising 1-2 wt. % of nitrogen sources and other suitable components in a fermentor or reactor;
b) adding 2 to 10 wt. % bacterial inoculum to the above fermentation medium;
c) incubating fermentation medium mixture of step b) at specific conditions followed by centrifugation; and
d) adding ice-cold solvent to the mixture of step c) to obtain the poly gamma glutamic acid.

In preferred embodiment of the present invention, the natural source used in step a) is selected from flower waste, Jaggery, molasses, or their combinations and the like.

In preferred embodiment of the present invention, the natural source used in step a) is in range of 20-55 wt. % or preferably is in range of 20-50 wt. % or more preferably is in range of 20-40 wt. % or even more preferably is in range of 20-30 wt. %.

In an embodiment of the present invention, the fermenter used in the present invention is stirred tank fermenter.

In another embodiment of the present invention, the pH of the fermentation medium is in a range of 6.5 to 7.5.

In further embodiment of the present invention, the pH of the fermentation medium is adjusted by using sodium hydroxide.

In another preferred embodiment of the present invention, the bacteria used for the inoculation step b) is selected from Bacillus species and more particularly Bacillus species is selected from Bacillus anthracis, Natrialba aegyptiaca, Bacillus subtili, Bacillus licheniformis, Bacillus megaterium, Staphylococcus epidermidis, Bacillus siamensis, and Bacillus paralicheniformis. More particularly the bacillus species is selected from Bacillus paralicheniformis.

In another preferred embodiment of the present invention, the nitrogen sources of the fermentation medium are obtained from materials selected from ammonium sulfate, ammonium chloride, ammonium citrate, ammonium acetate, and ammonium nitrate, or the like.

In another preferred embodiment of the present invention, the other suitable components of the fermentation medium are selected from citric acid, potassium hydrogen phosphate (K₂HPO₄), magnesium sulfate (MgSO₄), calcium chloride (CaCl₂), ferric chloride (FeCl₃), or the like.

In another preferred embodiment of the present invention, the incubation conditions are at a temperature in the range of 28-30° C. for a time period of 24-72 hrs.

In another preferred embodiment of the present invention, the solvent is selected from organic solvent and more particularly the solvent is lower alcohol. Preferably, the solvent is selected from methanol or ethanol.

The term lower alcohol as used herein refers to methanol, ethanol, propanol, isopropanol (isopropyl alcohol), butanol, isobutanol, and pentanol.

In an embodiment, the present invention provides a glutamic acid-independent process for the synthesis of poly gamma glutamic acid using jaggery, blackstrap molasses, and floral waste along with the use of nitrogen sources and bacteria in a fermentation medium. More, particularly the present invention discloses the use of Bacillus paralicheniformis NCIM 5769 for the synthesis of poly gamma glutamic acid.

The present invention provides the potential of jaggery, blackstrap molasses, and floral waste as substrate for poly gamma glutamic acid synthesis without the need of L-glutamic acid, thereby providing an economically efficient process for the synthesis of γ-PGA.

In another embodiment, the present disclosure provides a glutamic acid independent process for the synthesis of poly gamma glutamic acid from jaggery comprising the process of fermenting the media comprising Jaggery and ammonium nitrate by using Bacillus paralicheniformis NCIM 5769 at a specific reaction temperature in the range of 28° C. to 30° C. for time period of 24 to 72 h to obtain γ-PGA. Specifically, the process comprises:
a) preparing fermentation media by mixing jaggery with ammonium nitrate followed by adjusting pH to 7.5 using NaOH solution;
b) adding bacterium inoculum to the fermentation medium obtained in step (a), followed by agitation and aeration at 250 to 400 rpm and 1 vvm, respectively;
c) incubating the fermented medium at 28° C. for 72 h followed by centrifugation at 10000 rpm for 30 minutes; and
d) treating the supernatant of mixture with ice-cold methanol to precipitate the γ-PGA.

In an embodiment of the present invention, the pH of the fermentation medium is maintained in the range of 6.5 to 7.5.

In an aspect of the invention, the pH of the medium is adjusted to 6.5 to 7.5 by sodium hydroxide.

In an embodiment of the invention, Jaggery refers to an unrefined extract from sugarcane that has been concentrated to a solid. The sugarcane juice may be partially processed by filtration, clarification, boiling, crystallisation and/or centrifugation in the making of jaggery.

In an embodiment of the present invention jaggery can be selected from sugarcane jaggery palm tree jaggery, coconut jaggery or mixtures thereof.

In a preferred embodiment of the present invention, the source of nitrogen is inorganic source selected from ammonium sulfate, ammonium chloride, ammonium citrate, ammonium acetate, ammonium nitrate or mixtures thereof.

In another preferred embodiment of the present invention, the bacteria that can be used for the synthesis of poly gamma glutamic acid are selected from Bacillus species and more particularly Bacillus species are selected from Bacillus anthracis, Natrialba aegyptiaca, Bacillus subtili, Bacillus licheniformis, Bacillus megaterium, Staphylococcus epidermidis, Bacillus siamensis, and Bacillus paralicheniformis.

In another preferred embodiment, the present invention providing PGA production from jaggery facilitates a reduction in the cost of production associated with organic nitrogen source in case of synthetic medium. It has also been observed that Bacillus paralicheniformis produces about 200 g/L of γ-PGA, with productivity of 2.77 g/L/h from jaggery which is the highest to date using L-glutamic acid independent approach in batch fermentation.

In yet another preferred embodiment, the present invention provides a glutamic acid-independent process for the synthesis of poly gamma glutamic acid using jaggery, comprising fermenting the media comprising 50% Jaggery and 1.5% NH₄NO₃ using Bacillus paralicheniformis at 28° C. for 72 h to obtain 200 g/L of γ-PGA. The detailed processing steps are given below:
a) preparing a fermenting medium by mixing 50% jaggery with 1.5% of ammonium nitrate to adjust the pH to 7.5 in a fermenter;
b) adding 10% of inoculum to the fermentation medium obtained in step (a), followed by agitation and aeration at 250 rpm and 1 vvm respectively;
c) incubating the fermented medium at 28° C. for 72 h followed by centrifugation at 10,000 rpm for 30 min; and
d) adding four volumes of ice-cold methanol to the supernatant to precipitate the γ-PGA.

In another embodiment, the present invention provides, a glutamic acid independent process for the synthesis of poly gamma glutamic acid from floral extract comprising the steps of:
a) extracting floral waste by hot water extraction protocol which is followed by concentrating using Rota vapor to obtain a concentrate;
b) preparing a fermentation media with additional material(s) selected from ammonium nitrate, citric acid, K₂HPO₄, MgSO₄, CaCl₂), and FeCl₃;
c) mixing the concentrate of step (a) with the fermentation media of step (b) in an fermenter;
d) adding inoculum to the fermentation medium obtained in step (b), followed by agitation and aeration at 250 to 400 rpm and 1 vvm respectively;
e) incubating the fermented medium of step (d) at 28° C. to 30° C. for 24 h to 48 hrs by centrifugation at 10000 rpm for 30 min; and
f) adding ice-cold methanol to the supernatant to precipitate the γ-PGA.

In a preferred embodiment of the present invention, the pH of the fermentation medium is maintained in the range of 6.5 to 7.5.

In an embodiment of the present invention, the process of concentrating floral waste extract 2× times by using hot water extraction comprises steps of adding 100 g of floral waste into around 300 to 400 ml of water, followed by boiling at 100° C. for 1 to 2 hrs. Later it is passed through muslin cloth to collect the supernatant containing floral extract. This process is called hot water extraction. The floral extract is then concentrated (step a) using Rota vapor to which ammonium nitrate, citric acid, K₂HPO₄, MgSO₄, CaCl₂, and FeCl₃ are added.

In another preferred embodiment, the bacteria that can be used for the synthesis of poly gamma glutamic acid are selected from Bacillus species and more particularly Bacillus species are selected from Bacillus anthracis, Natrialba aegyptiaca, Bacillus subtilsi, Bacillus licheniformis, Bacillus megaterium, Staphylococcus epidermidis, Bacillus siamensis, and Bacillus paralicheniformis.

In yet another preferred embodiment of the invention, the floral waste means waste containing different types of wasted flower.

In yet another preferred embodiment, a glutamic acid independent process for the synthesis of poly gamma glutamic acid from flower comprising the use of fermenting media comprising of ammonium nitrate, 1%; citric acid, 1%; K₂HPO₄, 0.1%; MgSO₄, 0.05%; CaCl₂), 0.02%; FeCl₃, 0.003% using Bacillus paralicheniformis.

In yet another preferred embodiment, the present invention facilitates increase in γ-PGA production using floral waste, where the floral waste is effectively valorized to γ-PGA with the yield of 47 g/L with the productivity of 1.95 g/L/h within 24 h of fermentation at 28° C. using media composition comprising of floral extract concentrated 2 times (2×); ammonium nitrate, 1%; citric acid, 1%; K₂HPO₄, 0.1%; MgSO₄, 0.05%; CaCl₂, 0.02%; FeCl₃, 0.003%.

In yet another preferred embodiment of the invention, the floral waste is completely free of glutamic acid and sugars such as sucrose, glucose, and fructose which is naturally present in the floral waste contributes in the synthesis of γ-PGA.

In another embodiment, the present invention provides, a glutamic acid independent process for the synthesis of poly gamma glutamic acid from blackstrap molasses comprising the steps of:
a) preparing an inoculum;
b) preparing a production medium comprising of 10% to 30% molasses, and 1% to 2% ammonium nitrate;
c) adding the inoculum into the production medium followed by incubating at 28° C. to 30° C. for 24 h to 48 h, followed by maintaining agitation and aeration at 250-400 rpm and 1 vvm, respectfully to obtain a fermented broth;
d) centrifugating the fermented broth at 10000 rpm to remove the cells at 1 vvm to obtain a supernatant; and
e) adding four volumes of ice-cold methanol to the supernatant to precipitate the γ-PGA.

In a preferred embodiment of the present invention, the pH of the fermentation medium is maintained in the range of 6.5 to 7.5.

In another preferred embodiment, the present invention provides a glutamic acid independent process for the synthesis of highly pure poly gamma glutamic acid, wherein the bacteria Bacillus paralicheniformis NCIM 5769 is maintained in the medium comprising of (%): Beef extract, 0.1; Yeast extract, 0.2; Peptone, 0.5; NaCl, 0.5 and incubated at 28° C. for 24 h and this medium use as an inoculum.

In yet another preferred embodiment, the present invention provides a glutamic acid independent process for the synthesis of highly pure poly gamma glutamic acid from blackstrap molasses, comprising the steps of:
a) preparing an inoculum by maintaining the strain of Bacillus paralicheniformis NCIM 5769 in the medium comprising of (%): Beef extract, 0.1; Yeast extract, 0.2; Peptone, 0.5; NaCl, 0.5 and incubated at 28° C. for 24 h. The overnight grown culture was used as inoculum for the further studies.
b) preparing a production medium comprising of molasses, 10% to 30%; and ammonium nitrate, 1% to 2%;
c) adding 10% of the inoculum of step a) into the production medium of step (b) followed by incubating at 28° C. for 48 h to obtain a fermented broth;
d) centrifugation the fermented broth to remove the cells and obtain a supernatant; and
e) adding four volumes of ice-cold methanol to the supernatant to precipitate the γ-PGA.

In yet another preferred embodiment, the optimized medium comprising of 30% molasses and 2% ammonium nitrate yielded 132 g/L of γ-PGA.

In a nutshell, the present invention proves the potential of Jaggery, blackstrap molasses, and floral waste as an effective medium for L-glutamic acid independent production of PGA to completely substitute the synthetic production medium thereby decreasing the overall production cost in the synthesis of γ-PGA.

The present invention also entails fermentation approach for the highest production and productivity of γ-PGA in the batch fermentation.

The gene sequence of bacterium used in the present invention is provided in the text format and it is deposited in gene bank having deposition number MT138545 (accession no. MCC0196, strain designation-H6).

In an aspect of the present invention is based on the concept of “Minimum nutrition maximum yield”, wherein the final optimized medium comprised of 50% jaggery and 1.5% ammonium nitrate with the γ-PGA yield of 150 to 200 g/L with the productivity of 2.77 g/L/h. Similarly, for floral waste the final optimized medium consisted of floral extract concentrated 2 times (2×); ammonium nitrate, 1%; citric acid, 1%; K₂HPO₄, 0.1%; MgSO₄, 0.05%; CaCl₂), 0.02%; FeCl₃, 0.003% with the γ-PGA yield of 40 to 50 g/L with the productivity of 1.95 g/L/h and for molasses the final optimized medium comprised of 30% molasses and 2% ammonium nitrate with the γ-PGA yield of 120 to 140 g/L and a productivity of 2.75 g/L/h.

### EXAMPLES

Following examples are given by way of illustration therefore should not be construed to limit the scope of the invention.

Materials used: The jaggery for the purpose of this invention obtained from local market of Pune, 411016, Maharashtra, India, the Molasses was purchased from ifarmerscart, an online service provider in Maharashtra, India, and the floral waste was collected from Chaturshringi Temple located in Pune, 411016, Maharashtra, India. Further, the reagents ammonium nitrate and K₂HPO₄ were purchased from Merck, and the citric acid, MgSO₄ and CaCl₂) were purchased from Hi-media, India.

### Example 1. L-Glutamic Acid Independent Production of γ-PGA from Jaggery

2 to 20 wt. % of jaggery (natural source) was introduced into a fermentation medium comprising 1-2 wt. % of a nitrogen source (ammonium nitrate) in a fermenter. An inoculum of 10% of Bacillus paralicheniformis NCIM 5769 was inoculated in the above fermentation media as depicted below and incubated at 28° C. for 48 h:

TABLE 1

| Fermentation | | | | | | A6 |
|---|---|---|---|---|---|---|
| medium | A1 | A2 | A3 | A4 | A5 | (Control) |
| Jaggery | 2% | 4% | 8% | 16% | 20% | — |
| Ammonium | 1% | 1% | 1% | 1% | 1% | 1% |
| nitrate | | | | | | |
| Citric acid | 1% | 1% | 1% | 1% | 1% | 1% |
| K₂HPO₄ | 0.1% | 0.1% | 0.1% | 0.1% | 0.1% | 0.1% |
| MgSO₄ | 0.05% | 0.05% | 0.05% | 0.05% | 0.05% | 0.05% |
| CaCl₂ | 0.02% | 0.02% | 0.02% | 0.02% | 0.02% | 0.02% |
| FeCl₃ | 0.003% | 0.003% | 0.003% | 0.003% | 0.003% | 0.003% |
| Yield(g/L) | 5.18 | 13.79 | 33.83 | 61.32 | 77.66 | 0.0 |

After 48 h of fermentation, the γ-PGA was centrifuged at 10,000 rpm for 30 min to remove cell, and supernatant was collected to obtain a mixture. To this mixture, four volumes of ice-cold methanol was added to precipitate γ-PGA.

### 1.1. Effect of Increased Concentration of Jaggery on γ-PGA Production

20-60 wt. % of jaggery (natural source) was introduced into a fermentation medium comprising 1 wt. % of a nitrogen source (ammonium nitrate) in a fermenter. An inoculum of 10% of Bacillus paralicheniformis NCIM 5769 was inoculated in the following fermentation media and incubated at 28° C. for 48 h:

TABLE 2

| Fermentation | | | | | |
|---|---|---|---|---|---|
| medium | B1 | B2 | B3 | B4 | B5 |
| Jaggery | 20% | 30% | 40% | 50% | 60% |
| Ammonium | 1% | 1% | 1% | 1% | 1% |
| nitrate | | | | | |
| Citric acid | 1% | 1% | 1% | 1% | 1% |
| K₂HPO₄ | 0.1% | 0.1% | 0.1% | 0.1% | 0.1% |
| MgSO₄ | 0.05% | 0.05% | 0.05% | 0.05% | 0.05% |
| CaCl₂ | 0.02% | 0.02% | 0.02% | 0.02% | 0.02% |
| FeCl₃ | 0.003% | 0.003% | 0.003% | 0.003% | 0.003% |
| Yield(g/L) | 77.66 | 119.33 | 141.75 | 150.61 | 0.0 |

After 48 h of fermentation, the γ-PGA was centrifuged at 10,000 rpm for 30 min to remove cell, and supernatant was collected to obtain a mixture. To this mixture, four volumes of ice-cold methanol was added to precipitate γ-PGA.

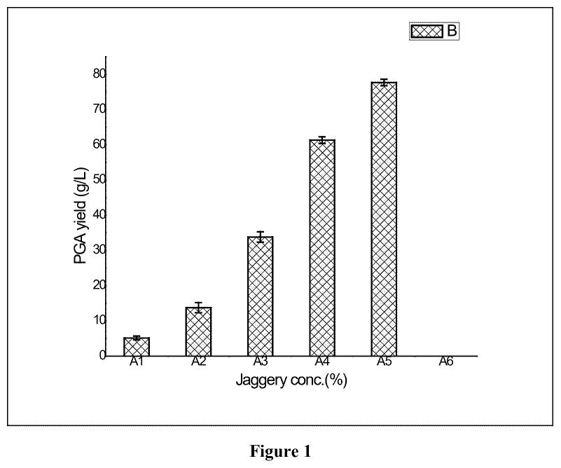

FIG. 1 clearly shows that as the concentration of jaggery was increased from 2% to 20% or more, the γ-PGA yield increased from 5 g/L to 78 g/L within 48 h of incubation.

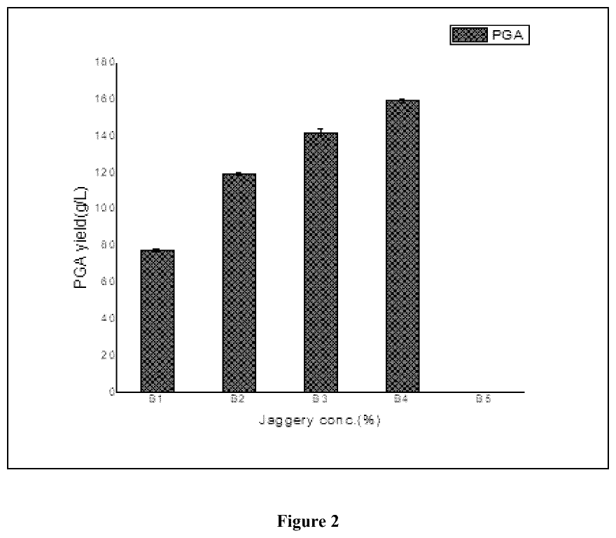

Increasing the concentration of Jaggery from 20% to 60%, elevated the γ-PGA production from 78 g/L to 150 g/L using 50% jaggery within 48 h of incubation. However, at 60% jaggery, no γ-PGA production was observed. Therefore, the optimum concentration of jaggery was found to be 50% with a yield of 150 g/L (FIG. 2).

Moreover, B5 column shows that at high concentrations of at or above 60% of Jaggery, did not give γ-PGA production at all.

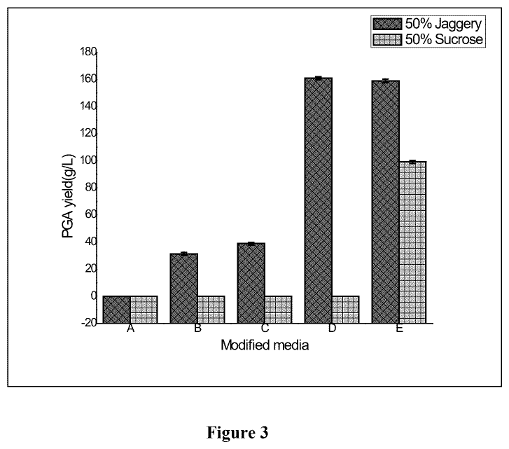

The experimental design was aimed to obtain a maximum yield of γ-PGA using minimum amount of nutrients. It was observed that 50% of jaggery along with citric acid yielded 30 g/L of γ-PGA within 72 h. Similarly, 50% of jaggery along with trace elements yielded 40 g/L of γ-PGA within 72 h. However, 50% jaggery in the presence of ammonium nitrate yielded 160 g/L of γ-PGA within 72 h. On the contrary, the control set containing 50% sucrose and ammonium nitrate did not show γ-PGA production, indicating that certain nutrient elements present in jaggery (other than sugars) aid in the production of γ-PGA. Similar results were observed using the 50% jaggery along with citric acid, ammonium nitrate, and trace elements with yield of 159 g/L. Therefore, based on this concept, the final optimized medium consisted of 50% jaggery and ammonium nitrate (FIG. 3).

1.2 Experimental Design for Minimum Nutrient and Maximum Yield Concept for PGA Production from Jaggery

50 wt. % of jaggery (natural source) was introduced into a fermentation medium comprising 1-2 wt. % of a nitrogen source (ammonium nitrate) in a fermenter. An inoculum of 10% of Bacillus paralicheniformis NCIM 5769 was inoculated in the following fermentation media and incubated at 28° C. to 30° C. for 24 to 72 h:

TABLE 3

| Fermen- | | | | | | A2 | B2 | C2 | D2 | E2 |
|---|---|---|---|---|---|---|---|---|---|---|
| tation | | | | | | (A1 | (B1 | (C1 | (D1 | (E1 |
| medium | A1 | B1 | C1 | D1 | E1 | control) | control) | control) | control) | control) |
| Jaggery | 50% | 50% | 50% | 50% | 50% | — | — | — | — | — |
| Ammoni- | | | | 1% | 1% | | | | 1% | 1% |
| um nitrate | | | | | | | | | | |
| Citric | | 1% | | | 1% | | 1% | | | 1% |
| acid | | | | | | | | | | |
| K₂HPO₄ | | | 0.1% | | 1% | | | 0.1% | | 0.1% |
| MgSO₄ | | | 0.05% | | 0.05% | | | 0.05% | | 0.05% |
| CaCl₂ | | | 0.02% | | 0.02% | | | 0.02% | | 0.02% |
| FeCl₃ | | | 0.003% | | 0.003% | | | 0.003% | | 0.003% |
| Sucrose | — | — | — | — | — | 50% | 50% | 50% | 50% | 50% |
| Yield | 0 | 31.39 | 39.15 | 160.94 | 159.01 | 0.0 | 0.0 | 0.0 | 0.0 | 99.17 |
| (g/L) | | | | | | | | | | |

After 72 h of fermentation, the γ-PGA was centrifuged at 10,000 rpm for 30 min to remove cell, and supernatant was collected to obtain a mixture. To this mixture, four volumes of ice-cold methanol was added to precipitate γ-PGA.

1.3 Effect of Increased Concentration of Ammonium Nitrate on Y-PGA production from jaggery

50 wt. % of a Natural Source was Introduced into a Fermentation medium comprising 0.5-2.5 wt. % of a nitrogen source (ammonium nitrate) in a fermenter. An inoculum of 10% of Bacillus paralicheniformis NCIM 5769 was 10 inoculated in the following fermentation media and incubated at 28° C.-30° C. for 24 to 72 h:

TABLE 4

| Fermentation | | | | | |
|---|---|---|---|---|---|
| medium | D1 | D2 | D3 | D4 | D5 |
| Jaggery | 50% | 50% | 50% | 50% | 50% |
| Ammonium | 0.5% | 1% | 1.5% | 2% | 2.5% |
| nitrate | | | | | |
| Yield | 107.48 | 160.14 | 200.65 | 135.13 | 136.46 |

### 1.4 Scale Up of γ-PGA Production in 1 L Fermenter Using Jaggery

The final optimized medium comprised of: Jaggery 50%; ammonium nitrate 1.5% and a pH of 7.5, wherein, 10% of the inoculum was added to the fermentation medium in the fermenter. The agitation and aeration were maintained at 250 rpm and 1 vvm respectively. After 72 h of incubation at 28° C. γ-PGA was extracted with four volumes of ice-cold methanol.

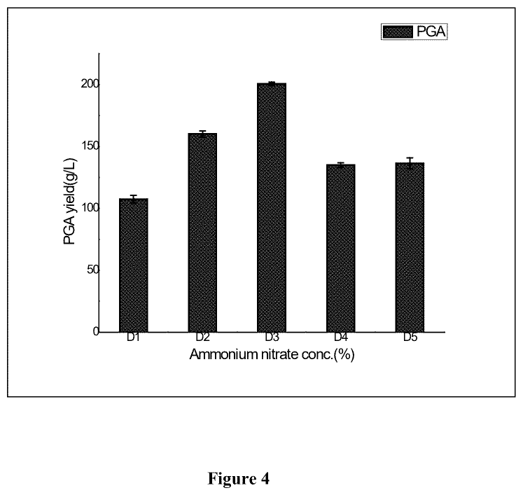

As the concentration of ammonium nitrate was increased from 0.5% to 1.5%, the yield of γ-PGA was elevated from 105 g/L to 200 g/L using 1.5% ammonium nitrate. Further increasing the ammonium nitrate concentration resulted in the decrease of γ-PGA yield. Therefore, the final optimized medium comprises of Jaggery 50% and ammonium nitrate, 1.5% with yield of 200 g/L within 72 h of incubation. Further, scale up to 1 L fermenter using this optimized medium showed similar reproducibility within 72 h (FIG. 4).

### Example 2. L-Glutamic Acid Independent Production of γ-PGA from Floral Waste

Sugars were extracted from floral waste by hot water extraction protocol (Waghmode et al., 2018). Briefly, 100 g of floral waste was added to 500 ml of water and heated at 100° C. for 2 h. Further it was passed through muslin cloth and the filtrate was collected and stored till further use. Inoculum of 10% of Bacillus paralicheniformis NCIM 5769 was inoculated in the following fermentation media and incubated at 28° C. for 24 h:

TABLE 5

| Fermentation | | | | | FEC |
|---|---|---|---|---|---|
| medium | FE1 | FE2 | FE3 | FE4 | (control) |
| Floral extract | 1X | 2X | 3X | 4X | Water |
| Concentra- | | | | | |
| tion | | | | | |
| Ammonium | 1% | 1% | 1% | 1% | 1% |
| nitrate | | | | | |
| Citric acid | 1% | 1% | 1% | 1% | 1% |
| K₂HPO₄ | 0.1% | 0.1% | 0.1% | 0.1% | 0.1% |
| MgSO₄ | 0.05% | 0.05% | 0.05% | 0.05% | 0.05% |
| CaCl₂ | 0.02% | 0.02% | 0.02% | 0.02% | 0.02% |
| FeCl₃ | 0.003% | 0.003% | 0.003% | 0.003% | 0.003% |
| Yield | 24 | 47 | 0.0 | 0.0 | 0.0 |

### 2.1 Scale Up of γ-PGA Production in 1 L Fermenter Using Floral Waste

The final optimized medium comprised of: Floral extract concentrated 2 times (2×); ammonium nitrate, 1%; citric acid, 1%; K₂HPO₄, 0.1%; MgSO₄, 0.05%; CaCl₂), 0.02%; FeCl₃, 0.003% and pH 7.5. 10% of the inoculum was added to the fermentation medium in the fermenter. The agitation and aeration were maintained at 250 rpm and 1 vvm respectively. After 24 h of incubation at 28° C. γ-PGA was extracted with four volumes of ice-cold methanol.

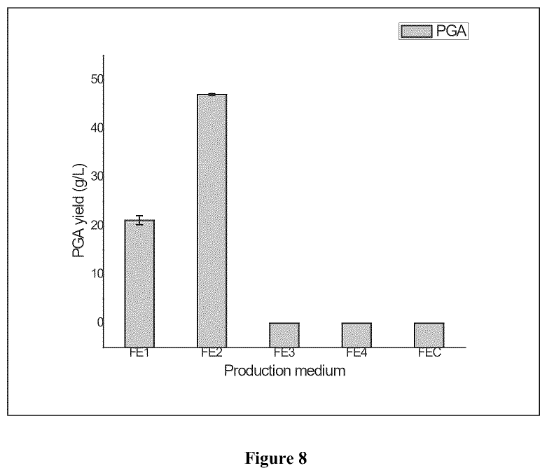

As floral waste was concentrated from 1× to 2×, the γ-PGA yield was elevated from 21 g/L to 47 g/L within 24 h. Further concentrating the floral extract resulted in a significant decrease in γ-PGA yield. Therefore, the optimized floral waste media consisted of floral extract concentrated 2 times (2×); ammonium nitrate, 1%; citric acid, 1%; K₂HPO₄, 0.1%; MgSO₄, 0.05%; CaCl₂), 0.02%; FeCl₃, 0.003% with a yield of 47 g/L and productivity of 1.95 g/L/h (FIG. 8). Further, scale up to 1 L fermenter using this optimized medium showed similar reproducibility within 24 h.

### 2.2 Purification of γ-PGA by Dialysis

1% of γ-PGA solution was prepared using deionized water and centrifuged at 10000 rpm for 30 min to remove any insoluble materials. The supernatant was desalted by dialysis (MW cut-off 14 kD) for 24 h followed by lyophilization to get pure material (Goto, A., & Kunioka, 1992).

### Example 3. Experimental Design for the “Minimum Nutrient with Maximum Yield” Concept for PGA Production from Molasses

The sole aim of this design was to obtain the maximum yield of γ-PGA using a minimum amount of fermentation medium components. To check the effect of molasses on γ-PGA production following media optimization was carried out. In the control set E, sucrose was used as a substitute for molasses.

TABLE 6

| Fermentation | | | | | | | | | | | | | | | |
|---|---|---|---|---|---|---|---|---|---|---|---|---|---|---|---|
| medium | A1 | A2 | A3 | B1 | B2 | B3 | C1 | C2 | C3 | D1 | D2 | D3 | E1 | E2 | E3 |
| Molasses | 20% | 25% | 30% | 20% | 25% | 30% | 20% | 25% | 30% | 20% | 25% | 30% | 20% | 25% | 30% |
| Ammonium | 1% | 1% | 1% | 1% | 1% | 1% | | | | | | | 1% | 1% | 1% |
| nitrate | | | | | | | | | | | | | | | |
| Citric acid | 1% | 1% | 1% | | | | 1% | 1% | 1% | | | | | | |
| K₂HPO₄ | 0.1% | 0.1% | 0.1% | | | | | | | 0.1% | 0.1% | 0.1% | | | |
| MgSO₄ | 0.05% | 0.05% | 0.05% | | | | | | | 0.05% | 0.05% | 0.05% | | | |
| CaCl₂ | 0.02% | 0.02% | 0.02% | | | | | | | 0.02% | 0.02% | 0.02% | | | |
| FeCl₃ | 0.003% | 0.003% | 0.003% | | | | | | | 0.003% | 0.003% | 0.003% | | | |
| Sucrose | | | | | | | | | | | | | 20% | 25% | 30% |
| Yield | 80.07 | 85.22 | 114.4 | 78.99 | 84.25 | 112.9 | 0 | 0 | 0 | 0 | 0 | 0 | 0 | 0 | 0 |

The inoculum of 10% was inoculated into following production media and incubated at 28° C. for 48 h. After 48 h, the fermented broth was centrifuged to remove the cell, followed by the addition of four volumes of ice-cold methanol to the supernatant to precipitate the γ-PGA.

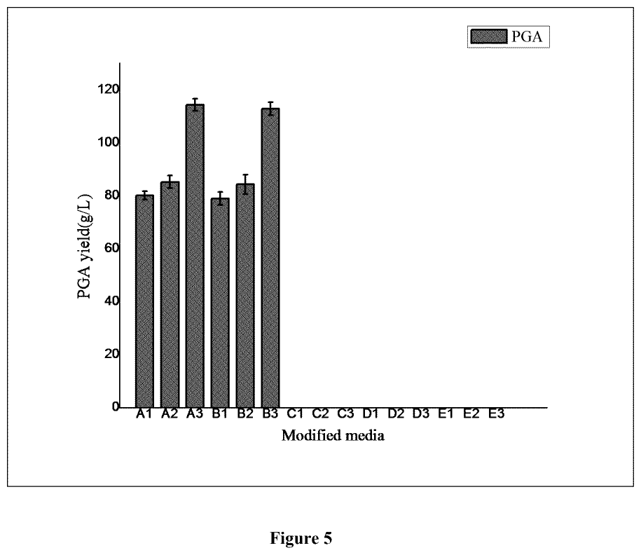

The ability of Bacillus paralicheniformis NCIM 5769 to utilize molasses with the least possible nutrients for γ-PGA production was evaluated. In modified medium A (A1, A2, A3), molasses was used as the carbon source in the presence of citric acid, ammonium nitrate, and trace elements (FIG. 5). As the concentration of molasses was increased from 20% (A1) to 30% (A3), the γ-PGA production elevated from 80 g/L to 114 g/L within 48 h. In modified medium B (B1, B2, B3), the ability of molasses to produce γ-PGA solely in the presence of ammonium nitrate was investigated. Increasing the concentration of molasses from 20% (B1) to 30% (B3), the γ-PGA yield increased from 78 g/L to 112 g/L within 48 h. In modified medium C (C1, C2, C3), the ability of molasses to produce γ-PGA solely in the presence of citric acid was investigated. However, no production of γ-PGA was observed within 48 h. In modified medium D (D1, D2, D3), the ability of molasses to produce γ-PGA solely in the presence of trace elements was investigated. However, no production of γ-PGA was observed within 48 h. To evaluate whether molasses was being used as a substitute for carbon source or was serving as a multi-nutritious waste medium, the γ-PGA production in modified medium E (E1, E2, E3) was investigated. Herein sucrose was used as a substitute for molasses. No production of γ-PGA was observed in the modified E medium, indicating that the molasses was being used as a multi-nutritious waste medium instead of carbon source alone by Bacillus paralicheniformis NCIM 5769. Therefore, based on this concept, the optimized medium consisted of 30% molasses and ammonium nitrate.

3.1 Effect of Ammonium Nitrate on L-Glutamic Acid Independent Production of γ-PGA from Molasses

The inoculum of 10% was inoculated into the production medium consisting of: Molasses, 10% to 30%; ammonium nitrate, 1% to 2% and incubated at 28° C. for 48 h. After 48 h, the fermented broth was centrifuged to remove the cell, followed by the addition of four volumes of ice-cold methanol to the supernatant to precipitate the γ-PGA.

### 3.2 Scale Up to 1 L Fermenter

The final optimized medium comprised of molasses, 30% and ammonium nitrate, 2%. Inoculum of 10% was added to the fermentation medium and incubated at 28° C. for 48 h. The agitation and aeration were maintained at 250 rpm and 1 vvm, respectively. After 48 h of incubation, the γ-PGA was extracted with four volumes of ice-cold methanol.

### Example 4: Effect of Bacterium Type on L-Glutamic Acid Independent Production of γ-PGA from Molasses

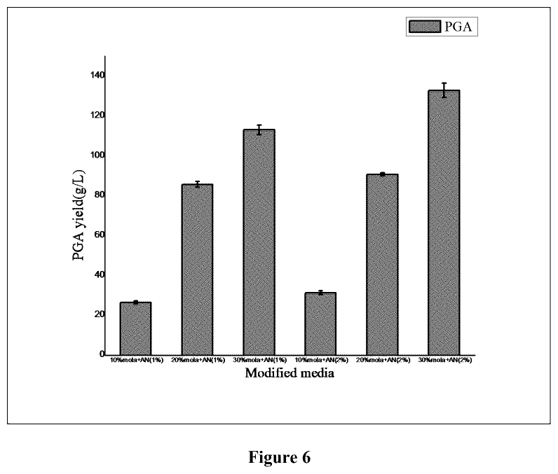

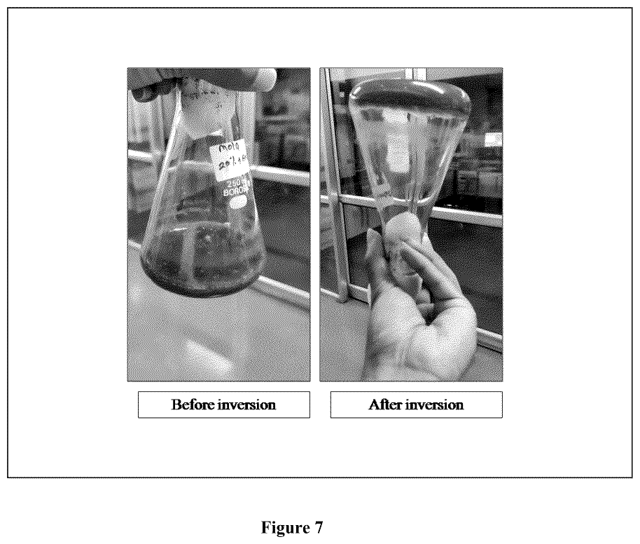

The ability of Bacillus paralicheniformis NCIM 5769 to produce γ-PGA in modified B medium containing an increasing concentration of ammonium nitrate was evaluated (FIG. 6). At ammonium nitrate concentration of 1%, the γ-PGA yield increased from 26 g/L (in 10% molasses) to 112 g/L (in 30% molasses) within 48 h. However, at 2% ammonium nitrate, the γ-PGA yield elevated from 31 g/L (in 10% molasses) to 132 g/L (in 30% molasses) within 48 h. The viscosity of the fermented broth was so high that it could pass the inversion test, as shown in (FIG. 7), indicating the extremely high production of γ-PGA. Therefore, the final optimized medium consisted of 30% molasses and 2% ammonium nitrate. This was so far the highest production of γ-PGA compared to both batch and fed-batch fermentation from molasses using L-glutamic acid independent feeding approach (refer Table 7). Finally, the scale-up of γ-PGA production to a 1 L fermenter using the final optimized medium showed similar reproducibility.

TABLE 7

| | | Yield | Productivity | |
|---|---|---|---|---|
| | Mode | (g/L) | (g/L/h) | Ref |
| Bacillus | Batch | 17.86 | 1.19 | Wang et |
| siamensis IR10 | fermentation | | | al., 2020 |
| | Fed-batch | 41.40 | 1.85 | |
| | fermentation | | | |
| B. subtilis NX-2 | Batch | 33.6 | 0.46 | Zhang et |
| | fermentation | | | al., 2012 |
| | Fed-batch | 51.1 | 0.56 | |
| | fermentation | | | |
| B. sutbilis NX-2 | Fed-batch | 84 | 1.27 | Feng et |
| | | | | al., 2016 |
| Bacillus | Batch | 132 | 2.75 | Present |
| paralicheniformis | fermentation | | | disclosure |
| NCIM 5769 | | | | |

### ADVANTAGES OF THE DISCLOSURE

1. Effective valorization of floral waste into a value added γ-PGA.
2. Process technology will help in the commercialization of γ-PGA
3. The present invention employs only natural sources such as floral waste and Jaggery as the substrate required to produce highly pure γ-PGA, therefore, γ-PGA shows 99% decrement of cost compared to conventional process that employ expensive media components.
4. The present invention provides a glutamic acid independent process for the synthesis of highly pure poly gamma glutamic acid and hence the cost involves is very less as compared to glutamic acid dependent process.
5. The media composition provided in present process consists of natural sources and specific organism which is osmophilic (e.g., B. paralicheniformis) and can sustain under high sugar concentration than known organisms like B. subtilis.
6. The present invention process uses 30% molasses which is 300 g/L and the yield of PGA obtained is 132 g/L which is 8 folds higher than known reports provided in background section.
7. The present invention provides use of osmophilic bacteria than can tolerate high molasses up to 300 g/L as a result of which high PGA production can be obtained.
8. This invention is an L-glutamic acid independent process which results in reduced production cost associated with use of glutamic acid.
9. Further the PGA yield obtained by batch fermentation of present invention is comparatively higher than both batch and fed batch approach disclosed by known reports disclosed in background section.

We claim:

1. A process for preparation of poly gamma glutamic acid using natural sources, the process comprising:
a) introducing 20-60 wt. % of a natural source into a fermentation medium comprising 1-2 wt. % of a nitrogen source in a fermenter or a reactor;
b) adding 2 to 10 wt. % of a bacterial inoculum to the fermentation medium of step a) to obtain a fermentation medium mixture;
c) incubating the fermentation medium mixture of step b) at temperature in a range 28-30° C. for a time period of 24-72 hrs followed by centrifuging the fermentation medium to obtain a mixture; and
d) adding an ice-cold solvent to the mixture of step c) to obtain the poly gamma glutamic acid.

2. The process as claimed in claim 1, wherein the natural source is selected from a jaggery, blackstrap molasses, floral waste, or mixtures thereof.

3. The process as claimed in claim 1, wherein the fermentation medium mixture further comprises citric acid, potassium hydrogen phosphate (K₂HPO₄), magnesium sulfate (MgSO₄), calcium chloride (CaCl₂)), ferric chloride (FeCl₃), or mixtures thereof.

4. The process as claimed in claim 1, wherein the nitrogen source is selected from ammonium nitrate, ammonium sulfate, ammonium chloride, ammonium citrate, ammonium acetate, or mixtures thereof.

5. The process as claimed in claim 1, wherein the bacterial inoculum is selected from Bacillus paralicheniformis, Bacillus anthracis, Natrialba aegyptiaca, Bacillus subtili, Bacillus licheniformis, Bacillus megaterium, Staphylococcus epidermidis, or Bacillus siamensis.

6. The process as claimed in claim 1, wherein the bacterial inoculum is Bacillus paralicheniformis NCIM 5769.

7. The process as claimed in claim 1, wherein the process is glutamic acid independent process.

8. The process as claimed in claim 2, wherein the jaggery is selected from sugarcane jaggery, palm tree jaggery, coconut jaggery, or mixtures thereof.

9. The process as claimed in claim 1, wherein pH of the fermentation medium is in a range of 6.5 to 7.5.

10. The process as claimed in claim 1, wherein the solvent is a lower alcohol.
